(19) United States
(12) Patent Application Publication
(10) Pub. No.: US 2017/0368281 A1
(43) Pub. Date: Dec. 28, 2017

(54) ARTIFICIAL-VENTILATION APPARATUS WITH CARDIAC MASSAGE MONITORING

(71) Applicant: Air Liquide Medical Systems, Antony (FR)

(72) Inventors: Marius DERMEL, Paris (FR); Eric JACQUOT, Antony (FR); Marceau RIGOLLOT, Montrouge (FR); Jean-Christophe RICHARD, Antony (FR)

(21) Appl. No.: 15/525,608

(22) PCT Filed: May 31, 2016
(86) PCT No.: PCT/FR2016/051298

§ 371 Date: May 10, 2017

(30) Foreign Application Priority Data
Jun. 8, 2015 (FR) 1555189

(51) Int. Cl. A61M 16/00; A61H 31/00

(52) U.S. Cl. CPC A61M 16/024; A61H 31/005; A61H 31/007; A61M 16/0069; A61M 2205/18; A61M 2016/0027; A61H 2201/107; A61M 2205/502; A61M 2205/52; A61M 2209/08; A61M 2016/0033

(57) ABSTRACT

The invention relates to a respiratory-aid apparatus (1) capable of supplying a stream of gas to a patient (P), comprising a gas-transport pipe (2) for transporting a stream of gas, such as air; measurement means (6) designed to measure at least one parameter representing the stream of gas and to supply at least one signal corresponding to said at least one parameter representing said stream of gas, for example the gas flow rate or pressure; signal-processing means (8) designed to process said at least one signal from the measurement means (6) and to deduce therefrom at least one piece of information (I1, I2, I3) characterising a cardiac massage performed on a patient; and display means (7) designed to display said at least one piece of information (I1, I2, I3) characterising a cardiac massage from the signal-processing means (8). The signal-processing means (8) are preferably capable of determining information representing the work (W_{V}, W_{T}) provided by the massage or pressure and/or flow rate amplitudes resulting from the massage. The invention also relates to a monitoring method capable of being implemented by such a respiratory-aid apparatus (1).

### CROSS REFERENCE TO RELATED APPLICATIONS

This application is a 371 of International PCT Application PCT/FR2016/051298 filed May 31, 2016 which claims priority to French Patent Application No. 1555189 filed Jun. 8, 2015, the entire contents of which are incorporated herein by reference.

### BACKGROUND

The invention relates to an artificial ventilation apparatus that can be used, during cardiac massage on a ventilated patient, in order to ensure monitoring of the cardiac massage performed by a first-aid provider, such as an emergency physician, a firefighter, a nurse or the like, and also to a monitoring method implemented by such an apparatus.

Certain respiratory assistance apparatuses are provided in emergency vehicles, such as ambulances, fire engines or emergency call-out vehicles, which attend road accidents, life-threatening emergencies or other dangerous situations. These respiratory assistance apparatuses are also called “ventilatory assistance apparatuses”, “artificial ventilation apparatuses” or, more simply, “medical ventilators”.

This is because emergency interventions often require the combination of mechanical ventilation and cardiac massage of the patient, particularly when the victim is in cardiac arrest, for example following an infarction.

Many studies show that the regularity of a cardiac massage performed on a person in cardiac arrest is a factor that determines its efficacy and, consequently, the chances of saving the patient in cardiac arrest. Thus, the general consensus usually recommends performing cardiac massage at a minimum frequency of 100 compressions per minute, with depression of the sternum by 4 to 5 cm.

However, when the massage is delivered manually, it is not humanly possible for a first-aid provider to comply with these recommendations throughout the duration of the massage procedure.

Furthermore, on account of the fatigue caused by manual cardiac massage, it is recommended that the persons performing the cardiac massage, that is to say the first-aid providers, change round every 2 minutes. However, this alternation between the persons providing the massage inevitably impacts on the regularity of the massage performed by them, since each first-aid provider logically adopts his or her own rhythm.

Devices do exist that show the massage provider the frequency and the depth of the massage. The most commonly used devices have to be fitted on the patient's chest.

However, a major disadvantage of these devices is that they add to all the equipment that the first-aid provider needs during his or her intervention and, what is more, fitting them takes up precious time, especially in cases of cardiopulmonary resuscitation, where time is of the essence.

The problem addressed is therefore to make available a medical respiratory assistance apparatus and a monitoring method by which it is possible to overcome all or some of the aforementioned problems and disadvantages, in particular a respiratory assistance apparatus that is able to assist a first-aid provider by allowing monitoring of one or more of the indicators representative of the cardiac massage performed on the patient, in such a way as to assist the first-aid provider in performing the cardiac massage, in particular by permitting the monitoring and display of an item of information relating to one or more characteristics of the cardiac massage, making it possible to maintain a regularity in the massage delivered, even in situations where the persons performing the massage change round.

### SUMMARY

The solution according to the invention thus concerns a respiratory assistance apparatus capable of supplying a flow of gas to a patient, comprising:
a gas delivery conduit for delivering a flow of gas, in particular air,
measuring means designed to measure at least one parameter representative of the flow of gas and to supply at least one signal corresponding to said at least one parameter representative of said flow of gas,
signal processing means designed to process said at least one signal originating from the measuring means and to deduce therefrom at least one item of information characteristic of a cardiac massage performed on a patient, and
display means designed to display said at least one item of information, characteristic of a cardiac massage, originating from the signal processing means.

Depending on the circumstances, the respiratory assistance apparatus of the invention can comprise one or more of the following technical features:
The measuring means are designed to measure at least one parameter representative of the flow of gas and chosen from between the pressure of the gas and the flowrate of the gas, in particular the flowrate of gas insufflated to the patient and/or the flowrate of gas exhaled by the patient.
The gas delivery conduit is supplied with gas from a gas source, which can be internal to the apparatus, in particular a motorized micro-blower, or external, in particular a gas reservoir, such as a gas canister, or a wall socket for delivery of gas supplied through a gas supply conduit.
The measuring means comprise at least one gas pressure sensor or a gas flowrate sensor.
The measuring means perform a measurement of at least one parameter representative of the flow of gas inside the gas delivery conduit.
According to a particular embodiment, the apparatus comprises a micro-blower feeding the gas delivery conduit, and the measuring means are designed to measure the speed of rotation of the motor of the micro-blower.
The signal processing means are able to determine an item of information representative of the work (W_{V}; W_{T}) provided by the massage or of the amplitudes of pressure and/or of flowrate resulting from the massage.
The signal processing means are configured to determine the amplitude of the variations in pressure or flowrate that are induced by a cardiac massage, by determining the difference between the maximum pressure (Pmax) or the maximum flowrate (Qmax), respectively, and the minimum pressure (Pmin) or the minimum flowrate (Qmin), that are reached during the phases of compression and relaxation of the thoracic cage of the patient.
The signal processing means are configured to determine an item of information representative of the work (W_{V}) provided by the massage, by calculating the integral of the ventilation pressure with respect to the volume of gas exchanged between the ventilator and the patient, during the compression phase of the cardiac massage and/or the relaxation phase of the cardiac massage. For example, the calculation is done using the following equation (I):

W_{V}=∫P·dV (I)

where:
W_{V} is the work provided by the massage
P is the ventilation pressure
dV is the derivative of the volume exchanged between the ventilator and the patient.
According to another embodiment, the signal processing means are configured to determine an item of information representative of the work (W_{T}) provided by the massage, by calculating the integral of the ventilation pressure with respect to time, during the compression phase of the cardiac massage and/or the relaxation phase of the cardiac massage.
   For example, the calculation is done using the following equation (II):

W_{T}=∫P·dT (II)

where:
W_{T} is the work provided by the cardiac massage
P is the ventilation pressure
dT is the derivative of the time.
The signal processing means comprise at least one electronic board, preferably an electronic board comprising at least one microprocessor using at least one algorithm.
It additionally comprises memorizing means for memorizing/storing information, data or the like, for example for storing the one or more items of information characteristic of the massage, which are determined by the signal processing means, and their changes over time.
The signal processing means are designed or configured to determine an item of information characteristic of the cardiac massage by applying one or more calculation algorithms to one or more signals representative of the gas flow, in particular pressure and/or flowrate signals, and the one or more calculation algorithms are preferably applied to a combination of signals representative of the gas flow. A calculation algorithm is understood as an algorithm configured to perform any mathematical operation, in particular calculations of the derivative, integral, addition, subtraction, multiplication or division type etc.
The signal processing means are configured, that is to say able and designed, to average the one or more items of information characteristic of the cardiac massage which are determined during one or more massage cycles, each cycle comprising a compression phase and a phase of relaxation (i.e. of decompression) of the thoracic cage of the patient.
The signal processing means are configured to calculate an average from the information gathered over several cycles, typically between one and several tens of cycles, for example an average obtained from at least 20 or 30 cycles, preferably at least 40 to 50 cycles.
The display means allow the user, that is to say the first-aid provider, to view said at least one item of information characteristic of a cardiac massage, in the form of values, curves, trends, etc. For example, the apparatus can display a value of work (W_{V}, W_{T}) provided by the massage, or a curve showing the change in this value, instantly updated with new values, or it can display the history of the change in this value over a given period of time that can be selected.
The display means comprise a screen for viewing the information, preferably a touch screen and/or color screen.
It additionally comprises alarm means by which the user is alerted when a determined and characteristic item of information (or several thereof) is outside a range of values that are predefined and/or memorized by the user, for example if it exceeds a high or maximum threshold value or, conversely, if it is less than a low or minimum threshold value.
The signal processing means are configured to control a triggering of the alarm means.
The alarm means comprise a visual alarm device and/or an acoustic alarm device, for example one or more constant or flashing warning lights on the information display screen and/or an acoustic signal output via a loudspeaker or the like.
It additionally comprises at least one battery or an electrical energy accumulator, preferably a rechargeable one, and/or a mains connection (110/230V).
The measuring means and the signal processing means are arranged in a cowling forming the external shell of the apparatus, said cowling additionally carrying the viewing means.
It comprises a connection to a gas outlet conduit, in fluidic communication with the gas delivery conduit, allowing the gas, such as air, to travel to a patient interface such as a breathing mask or a tracheal tube.

The apparatus of the invention thus makes it possible to monitor, in real time (or almost in real time), the cardiac massage that the first-aid provider or first-aid providers is/are in the process of performing on a patient who is in cardiac arrest, and, therefore, to be able to maintain regularity in the massage delivered, even in cases where the first-aid providers performing the massage change round.

The invention additionally relates to a monitoring method that is capable of being implemented by a respiratory assistance apparatus supplying a flow of gas, such as air or oxygen-enriched air, in particular the apparatus according to the invention as described above, in which method:

a) at least one parameter representative of a flow of gas within a gas delivery conduit is measured,

b) said measurement signal obtained at step a) is processed in order to deduce therefrom at least one item of information characteristic of a cardiac massage performed on a patient, typically a person who is in cardiac arrest, and

c) said at least one item of information characteristic of a cardiac massage, and obtained at step b), is displayed on display means.

Depending on the circumstances, the monitoring method according to the invention can comprise one or more of the following technical features:
The parameter representative of the flow of gas is chosen from between the pressure of the gas and the gas flowrate.
The parameter representative of the flow of gas is a flowrate of gas insufflated to the patient and/or a flowrate of gas exhaled by the patient.
The display at step c) is in the form of values or curves.
The parameter representative of the flow of gas measured at step a) is converted into at least one measurement signal representative of the flow of gas, prior to step b).
At step c), the item of information characteristic of the cardiac massage is representative of the work (W_{V}, W_{T}) provided by the massage.
At step c), the information characteristic of the cardiac massage is representative of the pressure amplitudes that are induced by the massage.
At step c), the information characteristic of the cardiac massage is representative of the flowrate amplitudes that are induced by the massage.
At step b), the amplitude of the variations in pressure that are induced by the cardiac massage are determined/evaluated by determining, for each compression and relaxation of the thoracic cage, the difference between the maximum pressure (Pmax) reached and the minimum pressure (Pmin) reached.
At step b), the amplitude of the variations in flowrate that are induced by the cardiac massage are determined/evaluated by determining, for each cycle of compression and relaxation of the thoracic cage, the difference between the maximum flowrate (Qmax) reached and the minimum flowrate (Qmin) reached.
At step c), an item of information characteristic of the cardiac massage and representative of the work (W_{V}) provided by the massage is determined/evaluated by calculating the integral of the ventilation pressure with respect to the volume (V) exchanged between the apparatus and the patient, during the compression phase of the cardiac massage and/or during the relaxation phase of the cardiac massage.
At step c), an item of information characteristic of the cardiac massage and representative of the work (W_{T}) provided by the massage is determined/evaluated by calculating, during the relaxation phase and/or compression phase, the integral of the ventilation pressure with respect to time (T).
At step c), an item of information characteristic of the cardiac massage is determined/evaluated by applying one or more calculation algorithms to one or more signals representative of the gas flow, preferably to a combination of signals representative of the gas flow, in particular signals concerning pressure (P) and flowrate (Q).
At step c), the one or more items of information characteristic of the cardiac massage which are determined during one or more massage cycles are averaged, the average being obtained either from several cycles or over a given period of time. For example, the average is obtained from at least 10 cycles, preferably at least 20 cycles, preferably at least 30 cycles, preferably at least 40 cycles, preferably at least 50 cycles. Alternatively, the average is obtained, for example, over a period of several seconds, preferably several tens of seconds, preferably over at least 40 seconds, more preferably at least 1 minute.

Generally speaking, within the context of the present invention, the item (or items) of information relating to one or more characteristics of the cardiac massage, and supplied to the first-aid provider, allows the latter to maintain regularity in the massage delivered, even in cases where the first-aid providers performing the massage change round.

### BRIEF DESCRIPTION OF THE DRAWINGS

The present invention will now be described in more detail with reference to the attached figures, in which:

FIG. 1 shows an embodiment of a respiratory assistance apparatus according to the present invention,

FIGS. 2A, 2B and 2C show operations that can be carried out, by a respiratory assistance apparatus according to the present invention, on the basis of the parameters that are measured in order to estimate an item of information characteristic of the cardiac massage,

FIG. 3 is an embodiment of a screen which shows items of information characteristic of the cardiac massage, and with which a respiratory assistance apparatus according to the present invention, such as the apparatus of FIG. 1, is equipped.

### DESCRIPTION OF PREFERRED EMBODIMENTS

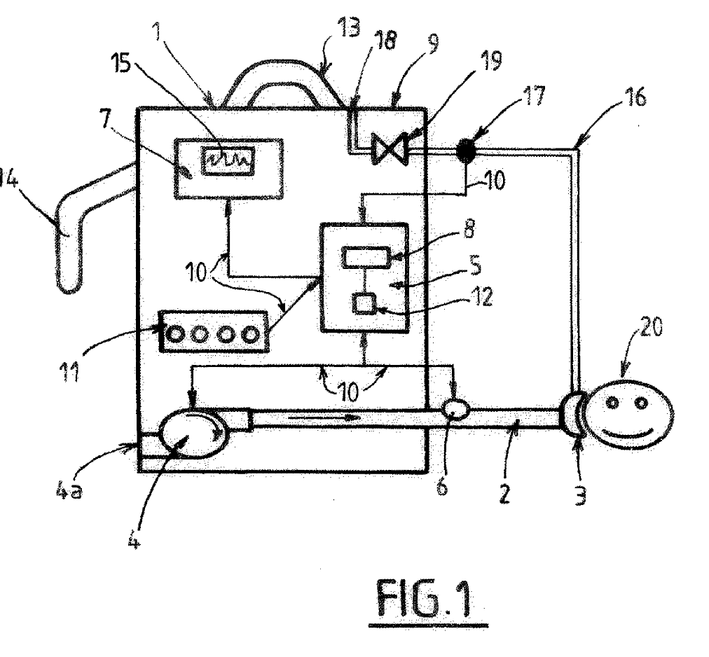

FIG. 1 is a diagrammatic representation of an embodiment of a ventilatory assistance apparatus or medical ventilator 1 according to the present invention.

It comprises a gas source 4, which is a motorized micro-blower here, also called a turbine, delivering a flow of respiratory assistance gas, typically a flow of air or of oxygen-enriched air. The air is sucked by the micro-blower via one or more inlet orifices 4a formed in the shell 9 of the ventilator 1.

In an alternative arrangement (not shown), the gas source 4 comprises a controlled valve supplied with gas, via an internal conduit, itself in fluidic communication with a gas reservoir or a wall socket for gas supply connected to a network of gas ducts, by way of a flexible conduit connecting the gas reservoir or the wall socket to the internal conduit.

The ventilatory circuit 2, 16, also called the patient circuit, comprising one or more passages, conduits or lines for gas, makes it possible to fluidically connect the gas source 4 of the ventilator 1 to the airways of a patient 20, by way of a patient interface 3, for example a breathing mask or an intubation tube.

The ventilatory circuit 2, 16 comprises at least one inhalation branch 2 for conveying the respiratory gas to the patient 20. It can also comprise an exhalation branch 16 designed to collect the gases exhaled by the patient 20, which are rich in CO₂. The exhalation branch 16 comprises an exhalation flowrate sensor 17, for example a hot-wire sensor, connected electrically to the control means 5, such as an electronic board, and also an exhalation valve 19 controlled by the control means 5. At its downstream end, the exhalation branch 16 communicates with the atmosphere via a gas outlet orifice 18, while its upstream end is connected to the inhalation branch 2, via a Y-shaped piece, i.e. directly to the patient interface 3.

Measuring means 6 are provided, such as a pressure and/or flowrate sensor, which are able and designed to measure at least one parameter representative of the flow of gas, in particular the pressure of the gas or the gas flowrate, whether the flowrate insufflated by the respirator and/or the gas flowrate exhaled by the patient 20, and to deliver at least one signal representative of said at least one measured parameter.

The measurement takes place in the inhalation branch 2 of the ventilatory gas circuit 2, 16 in such a way as to permit a measurement of the gas pressure or gas flowrate in said inhalation branch 2 serving as gas delivery conduit. In the embodiment illustrated in FIG. 1, the measuring means 6 are arranged outside the ventilator. However, according to another embodiment, they can also be located within the ventilator 1.

When the one or more parameters representative of the flow of gas have been measured, this measured parameter is converted into at least one signal representative of the flow of gas, which is then transmitted to and analyzed by signal processing means 8, that is to say typically the electronic board serving as control means 5, in order to deduce therefrom at least one item of information relating to a cardiac massage performed on a patient who is in cardiac arrest.

The signal processing means 8 thus form part of the control means 5 of the ventilator 1 and comprise one or more electronic boards with microprocessor(s) implementing one or more algorithms.

The ventilator 1 additionally comprises one or more control keys 11 which can be activated by the user, that is to say by the person performing the treatment, for example a first-aid provider, for example a stop/start button or the like, and also a screen 7 for viewing information, preferably a touch screen with color display.

When the signal processing means 8 have processed the one or more signals originating from the measuring means 6 and have deduced therefrom an item of information characteristic of a cardiac massage performed on the patient 20, this item of information is sent to and displayed by the display means 7, typically a touch screen.

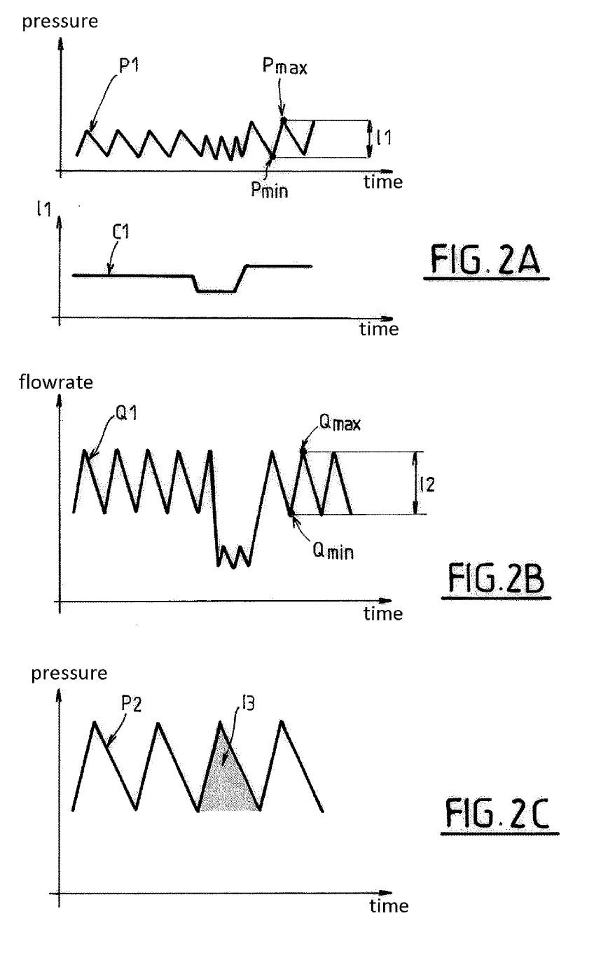

As is explained in detail below (FIGS. 2A-2C), the item of information characteristic of a cardiac massage can be a value of the work (W_{V}; W_{T}) provided by the massage or of the pressure and/or flowrate amplitudes resulting from the massage.

This item of information is displayed in the form of a value, or of a curve 15 showing a change, or a tendency to change, during a given period of time.

Memorizing means 12 make it possible to store all the items of information, data, tables, etc. For example, they comprise one or more flash memories or similar.

The transmission of the signals between the various components of the ventilator 1 is effected via suitable links, that is to say electrical links 10 such as cables or the like.

The ventilator 1 can also comprise a carrying handle 13 and/or a fastening system 14, such as a hook or the like, serving to hang the apparatus to a support. The ventilator 1 can also be transported inside a rigid case or a flexible bag.

FIGS. 2A-2C allow a better understanding of how the measurements are processed and which items of information I1, I2, I3 characteristic of a cardiac massage can be extracted from them.

Thus, FIG. 2A shows a graph of the ventilation gas pressure measured, during the performance of a cardiac massage, by the measuring means 6, such as a pressure sensor, of the respiratory assistance apparatus or ventilator 1 according to the invention.

The pressure sensor 6 makes it possible to measure gas pressures and to convert these measurements into signals which are transmitted to the information processing means 5, typically an electronic board, of the ventilator 1 which process them.

From these measured signals, the information processing means 5 of the ventilator 1 determine the maximum pressure Pmax reached by the ventilation pressure P1 during one or each compression phase and the minimum pressure Pmin reached by the ventilation pressure P1 during one or each relaxation phase of the cardiac massage.

The ventilator 1 then determines the item of information I1 characteristic of the cardiac massage, by calculating the difference between the maximum pressure Pmax and the minimum pressure Pm in during each compression/decompression cycle, where a compression/decompression cycle comprises a phase of compression followed by a phase of relaxation, that is to say of decompression.

Indeed, the ventilator according to the invention is designed and able to calculate this item of information I1 for all of the compression/decompression cycles of the cardiac massage.

The item of information I1 is in this case an amplitude of the variations of pressure P1 that are induced by the cardiac massage, corresponding to the difference between the maximum pressure (Pmax) and the minimum pressure (Pmin).

For its part, the curve C1 in FIG. 2A shows the change of the item of information I1 with respect to time, that is to say the change in the amplitude of the variations in pressure P1 during the cardiac massage.

The item of information I1 is then displayed on the screen of the ventilator 1 of the invention, preferably a color touch screen, so as to allow the first-aid provider to monitor these changes in amplitude of pressure P1 and, if necessary, to adapt the massage performed.

Similarly, FIG. 2B shows a graph of the flowrate Q1 of gas exchanged between the ventilator 1 and the patient, during the performance of a cardiac lo massage, and measured by the measuring means, such as a flowrate sensor, of the respiratory assistance apparatus or ventilator 1 according to the invention.

The flowrate sensor here makes it possible to measure gas flowrates and to convert these flowrate measurements into corresponding signals which are transmitted to the information processing means, typically an electronic board, of the ventilator 1 which process them.

More precisely, the information processing means determine, from these signals, the maximum flowrate Qmax reached by the flowrate Q1 of gas exchanged between the ventilator 1 and the patient, during the relaxation phase, and the minimum flowrate Qmin reached by the flowrate Q1 of gas exchanged during the compression phase of the cardiac massage.

They then deduce therefrom an item of information I2 characteristic of the cardiac massage, by calculating the difference between the maximum flowrate Qmax and the minimum flowrate Qmin, that is to say an item of information I2 corresponding to an amplitude of flowrate variations that are induced by the massage.

Here too, the ventilator of the invention is able and designed to calculate such an item of information I2 for all the compression and decompression cycles of the cardiac massage.

The item of information I2 is then displayed, as previously, on the screen of the ventilator 1 of the invention, in such a way as to allow the first-aid provider to monitor these changes in amplitude of flowrate and, if necessary, to adapt the massage performed.

In addition, FIG. 2C shows a graph of the ventilation pressure P2 measured, during the performance of a cardiac massage, by the measuring means, such as a pressure sensor, and in a manner similar to that explained above with reference to FIG. 2A.

Here, the item of information I3 characteristic of the cardiac massage is obtained by integrating the ventilation pressure P2 with respect to time for a cycle of compression and of relaxation and in fact corresponds to a work W provided by the massage.

The ventilator is capable of calculating this item of information I3, that is to say the work W, for all the compression and decompression cycles of the cardiac massage.

As has already been explained, the electronic board of the ventilator is able to perform a calculation of the integral of the ventilation pressure with respect:
either to the volume (V) of gas exchanged between the ventilator and the patient, during the compression phase of the cardiac massage and/or the relaxation phase of the cardiac massage, in such a way as to determine the “volume” work W_{V} provided by the massage,
or to time (T), during the compression phase of the cardiac massage and/or the relaxation phase of the cardiac massage, in such a way as to determine a “time” work W_{T} provided by the massage.

Here too, the item of information I3 is then displayed on the screen 7 of the ventilator 1 of the invention in such a way as to allow the first-aid provider to monitor the changes in the work W provided and, if necessary, to adapt the massage performed.

Generally speaking, the items of information I1, I2, I3 characteristic of a cardiac massage cannot only be displayed, as has already been explained, but can also be used to edit one or more reports that can be displayed on the screen 15 or that can be transferred by the ventilator for consultation by the user outside of the ventilator, for example for consultation on a computer, a smart phone, a tablet or on paper. This report contains the one or more items of information characteristic of the cardiac massage performed; it can also contain information concerning the characteristics of the ventilation delivered during the cardiac massage, for example the oxygen concentration of the gas supplied to the patient, the volume insufflated and exhaled by the patient, etc. These items of information can be displayed in the report in the form of values or curves.

Moreover, on the basis of the items of information I1, I2, I3 characteristic of a cardiac massage, the respiratory assistance apparatus 1 is able to generate visual and/or acoustic alarms. These alarms can alert the user to the fact that one or more items of information characteristic of a cardiac massage are outside a range of values that are predefined and/or stored by the user. For example, the range of predefined values associated with the item of information I1 contains a minimum value I1ₘᵢₙ and a maximum value I1ₘₐₓ. When the instantaneous or mean value of the item of information I1 is below the value I1ₘᵢₙ, a visual and/or acoustic alarm is generated and, by analogy, when the instantaneous or mean value of the item of information I1 is above the value I1ₘₐₓ, a visual and/or acoustic alarm is generated.

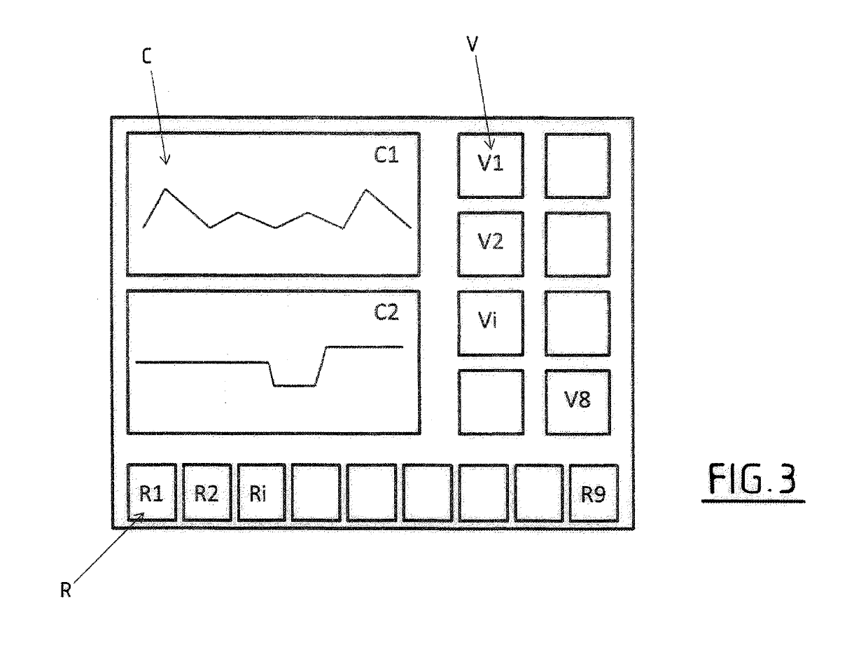

Moreover, FIG. 3 shows an embodiment of an information display screen 7 forming part of a respiratory assistance apparatus 1, that is to say a medical ventilator, according to the present invention, that can be used to administer a respiratory gas, for example air, to a person 20 who is in cardiac arrest, during the performance of cardiac massage.

The screen 7 is preferably a touch screen and is in color.

As will be seen, it displays information in the form of alphanumeric values in zones V of the screen and in the form of curves in zones C of the screen. The items of information displayed can be parameters characteristic of a cardiac massage or any other information determined by the respiratory assistance apparatus. For example, the item of information I1 characteristic of a cardiac massage is displayed in the zone V1, whereas an item of information representative of the volume of gas exchanged is displayed in the zone V2.

The zones C of the screen allow the change in an item of information over time to be displayed in the form of a curve. The item of information and its change, points constituting the curve, are extracted from the memorizing means 12. The items of information displayed can be parameters characteristic of a cardiac massage or any other information determined by the respiratory assistance apparatus. The length of time for which the item of information is shown is predefined or is selected by the user, and it is thus possible to display the change in the item of information over a greater or lesser duration.

The screen 7 can also display the ventilator controls R and can allow the user to modify them by pressing on the key Ri of the control that is to be modified.

Generally speaking, the artificial ventilation apparatus and the monitoring method implemented by such an apparatus are perfectly adapted to use during a cardiac massage on a ventilated patient, in order to ensure monitoring of the cardiac massage performed by the first-aid providers, such as an emergency physician, firefighter, nurse or the like.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1.-11. (canceled)

12. A respiratory assistance apparatus (1) capable of supplying a flow of gas to a patient (P), comprising:
a gas delivery conduit (2) for delivering a flow of gas,
a measuring device (6) designed to measure at least one parameter representative of the flow of gas and to supply at least one signal corresponding to said at least one parameter representative of said flow of gas,
a signal processing device (8) designed to process said at least one signal originating from the measuring device (6) and to deduce therefrom at least one item of information (I1, I2, I3) characteristic of a cardiac massage performed on a patient, and
a display device (7) designed to display said at least one item of information (I1, I2, I3), characteristic of a cardiac massage, originating from the signal processing device (8).

13. The apparatus of claim 12 wherein the measuring device (6) is designed to measure the at least one parameter representative of the flow of gas chosen from a gas pressure (P) and a gas flowrate (Q).

14. The apparatus as claimed in claim 12 wherein the measuring device (6) comprises at least one gas pressure sensor or at least one gas flowrate sensor.

15. The apparatus as claimed in claim 12 wherein the signal processing device (8) is capable of and configured to determine an item of information representative of a work (W_{V}, W_{T}) provided by the massage or of a pressure and/or a flowrate amplitude(s) resulting from the massage.

16. The apparatus of claim 12 wherein the signal processing device (8) is configured to determine a amplitude of a pressure or a flowrate variation that is induced by the cardiac massage, by determining a difference between a maximum pressure (Pmax) or a maximum flowrate (Qmax), respectively, and a minimum pressure (Pmin) or a minimum flowrate (Qmin), respectively, that are reached during a phase of compression and/or a phase of relaxation of a thoracic cage of the patient.

17. The apparatus of claim 12 wherein the signal processing device (8) is configured to determine an item of information representative of a work (W_{V}, W_{T}) provided by the cardiac massage, by calculating a integral of a ventilation pressure with respect:
either to a volume (V) of gas exchanged between the ventilator and the patient, during a compression phase of the cardiac massage and/or a relaxation phase of the cardiac massage,
or to a time (T), during a compression phase of the cardiac massage and/or a relaxation phase of the cardiac massage.

18. The apparatus of claim 12 wherein the signal processing device (8) comprises at least one electronic board comprising at least one microprocessor implementing at least one algorithm.

19. The apparatus of claim 12 wherein the display device (7) comprises a screen for viewing information.

20. A monitoring method that is capable of being implemented by a respiratory assistance apparatus (1) supplying a flow of gas the method comprising:
a) measuring at least one parameter representative of a flow of gas within a gas delivery conduit (2),
b) processing said measurement signal obtained at step a) to deduce therefrom at least one item of information (I1, I2, I3) characteristic of a cardiac massage performed on a patient (20), and
c) said at least one item of information (I1, I2, I3) characteristic of the cardiac massage, and obtained at step b), is displayed on a display device (7).

21. The method as claimed in claim 20, wherein the parameter representative of the flow of gas is chosen from a pressure of the gas and a gas flowrate.

22. The method as claimed in claim 20, wherein the display at step c) is in the form of one or more values or curves (15).
